(19) United States
(12) Patent Application Publication
(10) Pub. No.: US 2019/0083716 A1
(43) Pub. Date: Mar. 21, 2019

(54) INJECTION MOULDED NEEDLE ASSEMBLY

(71) Applicant: Novo Nordisk A/S, Bagsvaerd (DK)

(72) Inventor: Christian Saxe, Holte (DK)

(73) Assignee: Novo Nordisk A/S, Bagsvaerd (DK)

(21) Appl. No.: 15/766,476

(22) PCT Filed: Oct. 7, 2016
(86) PCT No.: PCT/EP2016/073948

§ 371 Date: Apr. 6, 2018

(30) Foreign Application Priority Data
Oct. 9, 2015 (EP) 15189186.8

(51) Int. Cl. A61M 5/32; A61M 5/34

(52) U.S. Cl. CPC A61M 5/3202; A61M 5/3275; A61M 2005/3206; A61M 5/3293; A61M 5/343

(57) ABSTRACT

A needle assembly for a medical device, comprising a needle hub adapted for receiving a needle cannula, and a needle shield having an open end. The needle assembly is adapted to be changed from an integral configuration, wherein the needle hub is aligned with the needle shield, wherein the first end of the needle shield is covered by the needle hub, and wherein a frangible connection forms an integral connection between a shield connecting portion and a hub connecting portion, to a broken configuration, wherein the frangible connection is broken, and whereby the needle hub and needle shield are two separate parts, and whereby the needle shield is removable from the needle hub.

### TECHNICAL FIELD

The invention relates to an injection moulded needle assembly and a method of producing an injection moulded needle assembly. The invention further relates to an injection moulded needle assembly comprising an integrally formed needle shield, and a method for producing an injection moulded needle assembly comprising an integrally formed needle shield.

### BACKGROUND

A conventional needle assembly comprises a needle hub, a needle cannula, an inner cap to protect the needle, and a container with a sealing paper to preserve sterility of the assembly during storage. Furthermore the container is rotationally locked to the hub, and may therefore be used as a mounting tool, to mount and demount the needle hub from a needle mount on an injection device.

A needle unit, e.g. a pen needle, is to be mounted on the needle mount of an injection pen. After injection, some users replace the inner cap on the hub, in order to cover the needle, and they furthermore put on a pen cap, in order to cover and protect the entire needle assembly mounted on the needle mount. In order to minimize the number of components of the needle assembly it has been suggested that the container should provide the function as container and an inner cap. However, the conventional container does not fit into the space between a hub mounted on a needle mount and a pen cap, and the container can, therefore, not provide the function of the inner cap, in this case.

In order to solve this problem it is known from US2009/069752 to provide a frangible line or frangible zone between a main portion and a removable flange. The removable flange provides a sealing surface to be attached to a sealing paper. The container can be used to accommodate the needle in a sterile condition before the removable part is removed. When the user breaks of the removable part, and removes it along with the sealing paper, the container may provide the function of the inner cap. However, as the container still is rather large, it may not fit under all types of pen caps.

On the injection device market there are several different types of injection devices, and it is desired to develop a container which can accommodate the needle in a sterile condition, and a container which can provide the function of an inner cap.

US 2012/0277685 allegedly discloses a safety needle and a needle assembly provided with a needle shield movably mounted on a hub so as to selectively cover the distal end of a needle. An interface is defined between the hub and the shield, and the interface is sufficiently sealed by portions of the hub or the shield, so as to define a sterility barrier. The needle assembly may be packaged for shipping and transportation without any external casing or packaging. The needle shield is mounted on an outer surface of the hub, is having an open end to permit passage of a needle.

U.S. Pat. No. 3,073,307 allegedly discloses a needle assembly formed by injection moulding. US 2013/0204197 and WO 2011/139229 discloses needle assemblies having frangible connections.

Having regard to the above, it is an object of the present invention to provide a cost-effective needle assembly with a minimum of components. The needle assembly should be easy to handle, easy to store in an initial sterile condition as well as during use.

### SUMMARY

In the disclosure of the present invention, embodiments and aspects will be described which will address one or more of the above objects or which will address objects apparent from the below disclosure as well as from the description of exemplary embodiments.

In a first aspect of the invention a needle assembly for a medical device is provided, wherein the needle assembly is having a central cylindrical axis and a radial axis defining a cylindrical and a radial direction, and wherein the needle assembly comprises a needle hub adapted for receiving a needle cannula, wherein the needle hub is having an inner and an outer surface, a proximal and a distal end.

The needle hub comprises a skirt portion comprising a connecting member for removably connecting the needle hub to a needle mount of the medical device, a proximal end defining an opening adapted to be sealed by a removable sealing member, and a distal end defining an opening adapted to be covered by a central hub portion.

The central hub portion comprises a through hole defining a central cylindrical axis collinearly aligned with the cylindrical axis, and is adapted to receive the needle cannula. The central hub portion further comprises a skirt connecting portion connected to a distal portion of the skirt portion.

The needle assembly also comprises a needle shield having a first end defining a first opening and a second end defining a second opening, and wherein the second end is adapted to be sealed by a removable sealing member, wherein the needle shield comprises a tubular portion defining a central cylindrical axis of the needle shield, and wherein the needle shield comprises an inner surface and an outer surface.

Furthermore, the needle hub comprises a shield connecting portion adapted for forming a first connection portion for a frangible connection to the needle shield, and the needle shield comprises a corresponding hub connecting portion adapted for forming a second connection portion for the frangible connection to the needle hub.

The needle assembly is adapted to be changed from an integral configuration, wherein the central cylindrical axes of the needle hub and the needle shield are collinearly aligned with the cylindrical axis, wherein the first opening defined at the first end of the needle shield is covered by the needle hub, and wherein the frangible connection forms an integral connection between the shield connecting portion and the hub connecting portion, and wherein a clearance (95) is provided between the needle shield (60, 160, 260, 360). The needle assembly can be changed to a broken configuration, wherein the frangible connection is broken, and whereby the needle hub and needle shield are two separate parts, and whereby the needle shield is adapted to be removable from the needle hub by pulling the needle hub (10, 110, 210, 310) and needle shield (60, 160, 260, 360) in opposite directions.

In this way, the needle shield is adapted to provide a clearance between the inner surface of the needle shield and the cylindrical axis, and the clearance is adapted to minimize the risk for the needle shield interfering with a received needle cannula.

Due to the second opening of the needle shield it is possible, in a one-part injection moulded needle assembly to provide a clearance between the needle and the inner surface of the needle shield. In other words, the second opening of the needle shield allows the forming of a clearance between the needle cannula and the inner surface of the needle shield. In some alternatives the aspect ratio between the clearance and the diameter of the tubular portion of the hub, may be in the range of 0.1 to almost 0.5, i.e., the clearance can extend from the needle cannula to the skirt portion. Such a clearance would not be possible if the mould insert was to be withdrawn from the open end of the hub portion.

The above described needle assembly provides a one part needle hub, which can be manufactured by injection moulding, which in a sealed condition can provide a sterile barrier for the needle cannula, and the needle assembly can be easily operated. By applying a torque or an axial force to the needle shield relative to the needle hub, the needle assembly can be changed from the integral to the broken configuration, wherein the frangible connection is broken. In the broken configuration, the needle hub and needle shield are two separate parts, and the needle shield can be removed from the needle hub, by pulling the hub and the shield in opposite directions relative to each other. This allows a user to use the needle assembly as a traditional pen needle assembly, where the cap is removed during use.

The needle shield is adapted to provide a clearance between the inner surface of the needle shield and the cylindrical axis, and the clearance is adapted to minimize the risk for the needle shield interfering with a received needle cannula. During use, there is a reduced risk of the needle shield interfering with a zone around the central axis, and as the needle to be received occupies a portion of the zone around the central axis, the described needle assembly enables a design reducing the risk of bending the needle.

A needle shield moulded with an open end is also more flexible than a needle shield moulded with a closed end, this will allow a design where some surface structures may be provided on the inner surface of the needle shield, e.g., a snap locking feature.

The shield connecting portion may be a surface portion occupying a portion of the outer surface of the needle hub, and the hub connecting portion may be a surface portion occupying a portion of the surface of the needle shield. The connecting portions are defined by the mould used to form the needle assembly, and the connecting portions will be formed on each side of the small channel forming the frangible zone.

In a further aspect, the needle shield is adapted to release a mould insert through the opening at the second end, and the inner surface of the shield has been formed by the mould insert.

Also or alternatively, the cylindrical axis and the inner surface of the needle shield provide a draft angle to ease the release of the mould insert. The draft angle is preferably between 0.2 and 5 degrees.

Also or alternatively, the inner surface of the needle shield defines a closed curve on a plane defined by a cylindrical coordinate along the cylindrical axis, wherein the cylindrical coordinate is defined between the first and the second end of the needle shield, wherein the closed curve defines a diameter dependent on the cylindrical coordinate, and wherein the diameter of the closed curve substantially increases in a direction from the first to the second end, whereby no volume is entrapped in the needle shield, i.e., there is no formation of under-cuts.

Also or alternatively, the needle shield comprises a first inner diameter at the opening of the first end, wherein the first inner diameter of the needle shield is larger than an inner diameter of the through hole of the needle hub, and thereby providing a needle assembly reducing the risk of bending a received needle.

Also or alternatively, the needle hub comprises a glue reservoir collinearly aligned with the through hole, wherein an inner surface of the glue reservoir defines a largest inner diameter at the distal end of the needle hub, and wherein the first inner diameter of the needle shield is larger than the largest inner diameter defined by the inner surface of the glue reservoir. In this way, the needle assembly is adapted to be manufactured by injection moulding, and in order to make a sufficient clearance between the needle cannula and the needle shield, the needle shield is formed with an open end, which allows the release and ejection of a mould, wherein the mould forms the inner surface of the needle shield.

In another aspect, the needle assembly is adapted to have a design providing slim construction, i.e. a small outer diameter. This is provided as the tubular portion of the needle hub defines a distal outer diameter at the distal end, wherein the shield connecting portion is a portion of the central hub portion and defines a shield connecting diameter, and wherein the shield connecting diameter is smaller than the distal outer diameter of the tubular portion.

Also or alternatively, the needle shield defines a first outer diameter at the first end, and wherein the first outer diameter of the needle shield is smaller than the distal outer diameter of the tubular portion of the needle hub.

Also or alternatively, the tubular portion of the needle hub defines a distal outer diameter at the distal end, wherein the shield connecting portion is a portion of the tubular hub portion and defines a shield connecting diameter, and wherein the shield connecting diameter is equal to or larger than the distal outer diameter of the tubular portion.

A slim construction of the needle assembly allows the shield to fit under a cap for a medical device, in a situation where it is desired to leave the needle assembly mounted on a needle mount and with the cap mounted on the medical device. Thereby, the needle is both protected by the cap and the needle shield.

In another aspect, the needle is provided with projecting hub portion to enable more functionality to the construction. This is provided by a needle assembly, wherein the central hub portion comprises a projecting hub portion having a tubular portion and a central portion covering a distal end of the tubular portion, wherein the through hole and the shield connecting portion is positioned on a distal portion of the projecting hub portion.

Also or alternatively, the shield connecting portion is positioned on an outer surface of the tubular portion of the projecting hub portion, wherein the opening at the first end of the needle shield is adapted to receive a portion of the projecting hub portion, and wherein the needle assembly is adapted to be changed from the integral configuration to the broken configuration by pushing the needle shield in the direction of the needle hub.

Also or alternatively, the first end of the needle shield is adapted to be pressure fitted onto the projecting hub portion, for the needle assembly being in the broken configuration.

The projecting portion provides an additional longitudinal surface on the tubular portion of the projecting portion, and this surface allows the provision of different functionalities relating to attachment between hub and shield.

In another aspect, the needle assembly provides a functionality of attaching the needle shield to the hub, after the frangible zone has been broken. This functionality may be provided by the opening at the second end of the needle shield, being adapted to receive a portion of the needle hub, wherein the inner surface at the second end of the needle shield is adapted to match an outer surface of the needle hub, and wherein the needle shield is adapted to be fitted onto the needle hub, by receiving the portion of the needle hub through the opening at the second end of the needle shield.

Also or alternatively, the inner surface of the needle shield forms a snap connecting member, and wherein an outer surface of the needle hub comprises a corresponding snap connecting member, and wherein the needle shield is adapted to be snapped onto the needle hub, for the needle assembly in the broken configuration.

Also or alternatively, the snap connecting member is positioned on an outer surface of the tubular portion of the projecting hub portion.

Also or alternatively, the snap connecting member is positioned on an outer surface of the tubular portion.

Also or alternatively, the cylindrical axis and the inner surface of the needle shield defines a draft angle enabling removal of a mold insert through the second end of the shield, and wherein the outer surface of the tubular portion of the projecting hub portion is adapted to match the draft angle and for forming a pressure fitted connection, whereby the connection between the second end of the needle shield portion and the projecting hub portion is optimized for forming a pressure fitted connection.

In another aspect of the invention, the needle assembly provides a construction with increased surface area for attaching a sealing member by welding. This is provided by the needle hub comprising a sealing flange positioned at the proximal end, which sealing flange is adapted to receive a sealing member, which is to be welded onto a surface.

Also or alternatively, the needle shield comprises a sealing flange positioned at the second end of needle shield, wherein the sealing flange is adapted to receive a sealing member, which is to be welded onto a surface.

In a further aspect, the needle assembly comprises structures that are manufactured by a needle mould which can be separated along two different directions, i.e., along the cylindrical axis and along the radial axis. This constrained is provided by a needle assembly, wherein the outer surface of the needle assembly comprises two or more radially extending structures, wherein the at least two radially extending structures confines a volume which is closed in the cylindrical direction. Also or alternatively, the radially extending structures comprise gripping structures.

In another aspect is provided, a needle assembly for a medical device (900) comprising a needle cannula and a needle hub, wherein the needle hub is having a skirt portion and a central portion, wherein the needle cannula is received in the central portion. The assembly further comprises a tubular shield adapted to shield a distal end of the needle cannula, and to cover a portion of the needle hub. The needle assembly also comprises a frangible zone, and the needle hub comprises a distal portion with a distal end, and a proximal portion with a proximal end having a first opening surrounded by a first sealing surface adapted to attach to a first sealing member. The distal portion is positioned on the central portion, and wherein the proximal portion of the needle hub is a portion of the skirt portion. The needle shield comprises a first portion having a first end and a second portion having a second end, and wherein the second end is having a second opening surrounded by a second sealing surface adapted to attach to a second sealing member. The second opening is adapted to be fitted onto a distal portion of the needle hub, and thereby being adapted to shield the needle cannula. The needle assembly is adapted to be changed from an initial shielded configuration, wherein the frangible zone directly connects the distal portion of the hub and the first portion of the needle shield, and a broken configuration, wherein the frangible zone is broken and the needle shield s removable from the needle hub.

In another aspect is provided a medical system comprising a needle assembly as described above, and a medical device comprising:
a mounting portion,
a device cap adapted to cover the mounting portion, and
wherein the mounting portion comprises a needle mount and a device cap mount,
   wherein the medical system is configured to be in a state:
wherein the needle assembly is mounted on the needle mount,
wherein the needle assembly is in a broken configuration,
wherein the needle shield is fitted onto a distal portion of the needle hub,
wherein the device cap is mounted on the cap mount, and
wherein the device cap accommodates the needle assembly.

In another aspect is provided, a method of handling a needle assembly as described above, wherein the method comprises:
providing a needle assembly,
changing the needle assembly from an integral configuration to a broken configuration.

In a further aspect the needle shield is fitted onto the needle hub, when the needle assembly is in the broken configuration.

In another aspect is provided a method of producing a needle assembly as described above, wherein the method comprises:
providing an injection mould for forming the needle assembly
filling the injection mould and thereby forming the needle assembly
pulling the injection mould in a first direction, and thereby separating the mould from a first portion of the needle assembly
separating the injection mould along a second direction, and thereby separating the mould from a second portion of the needle assembly.

### DESCRIPTION OF DRAWINGS

In the following the invention will be further described with reference to the drawings, wherein:

FIG. 1(a) shows a needle assembly comprising a needle hub and a needle shield, wherein the needle assembly is in an integral configuration.

FIG. 1(b) shows the needle assembly from FIG. 1(a) in an integral configuration and a medical device with a needle mount, and wherein a first end of the needle shield is connected to the needle hub.

FIG. 1(c) shows the needle assembly and the medical device from FIG. 1(b), wherein the needle assembly is mounted on the needle mount in an integral configuration.

FIG. 1(d) shows the needle assembly and the medical device from FIG. 1(c), wherein the needle assembly is mounted on the needle mount in a broken configuration, and wherein the needle shield has been removed from the needle hub.

FIG. 1(e) shows the needle assembly and the medical device from FIG. 1(d), wherein the needle assembly is mounted on the needle mount in a broken configuration, and wherein the needle shield has been returned on the needle hub, and wherein a second end of the needle shield is connected to the needle hub.

FIG. 2(a) shows another embodiment of a needle assembly comprising a needle hub and a needle shield, wherein the needle assembly is in an integral configuration, and wherein a first end of the needle shield is connected to the needle hub.

FIG. 2(b) shows the needle assembly from FIG. 2(a) mounted on a needle mount of a medical device in a broken configuration, and wherein the needle shield is connected to the needle hub through a first end.

FIG. 3(a) shows another embodiment of a needle assembly comprising a needle hub and a needle shield, wherein the needle assembly is in an integral configuration.

FIG. 3(b) shows another embodiment of a needle assembly comprising a needle hub and a needle shield, wherein the needle assembly is in an integral configuration.

In the figures like structures are mainly identified by like reference numerals.

### DESCRIPTION

When in the following terms such as “upper” and “lower”, “right” and “left”, “horizontal” and “vertical” or similar relative expressions are used, these only refer to the appended figures and not to an actual situation of use. The shown figures are schematic representations for which reason the configuration of the different structures as well as there relative dimensions are intended to serve illustrative purposes only.

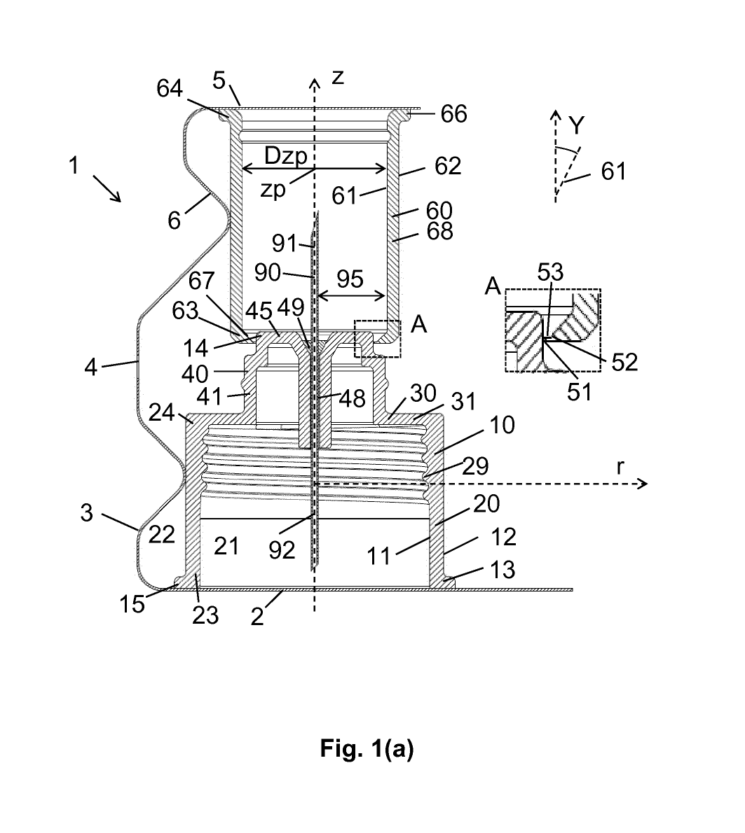

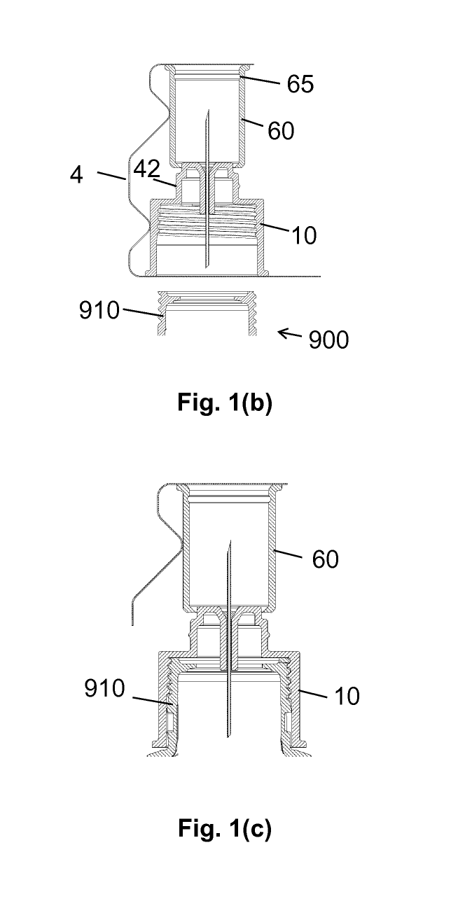

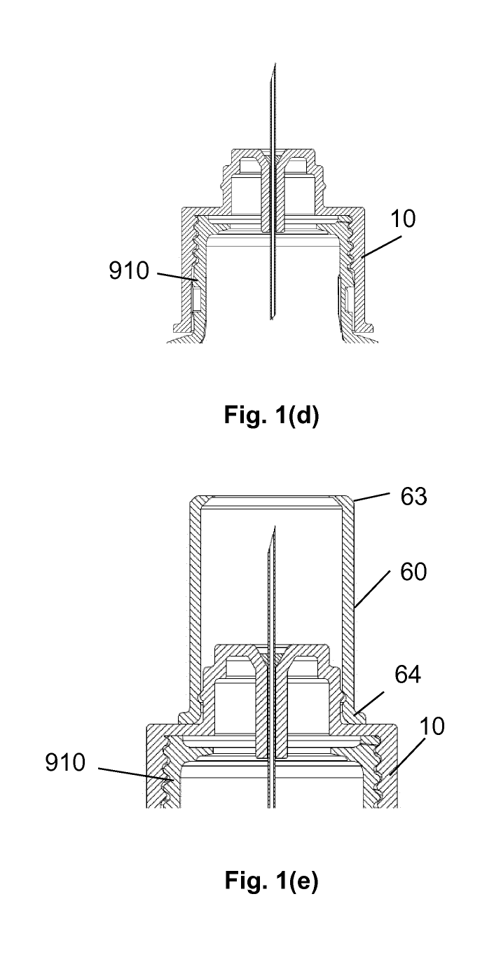

FIG. 1(a) shows a needle assembly 1 for a medical device 900. The figure also shows a cylindrical axis (z) along a central longitudinal axis of the medical device, and a radial axis (r) being orthogonal to the cylindrical axis. The cylindrical axis defines a cylindrical or longitudinal direction, and the radial axis defines a radial or transverse direction.

The needle assembly comprises a needle hub 10 adapted for receiving a needle cannula 90, wherein the needle hub 10 is having an inner surface 11 and an outer surface 12. The hub 10 further defines a proximal end 13 and a distal end 14.

The needle hub also comprises a skirt portion 20 defining a circumference of the needle assembly, and a connecting member 29 for removably connecting the needle hub 10 to a needle mount 910 of the medical device 900. The skirt portion 20 is having a proximal end 23 defining an opening adapted to be sealed by a removable sealing member 2, and a distal end 24 defining an opening adapted to be covered by a central hub portion 30. The central hub portion 30 comprises a through hole 48 collinearly aligned with the central axis, and adapted to receive the needle cannula 90, and a skirt connecting portion 31 connected to a distal portion of the skirt portion 20.

FIG. 1(a) also shows a needle shield 60 having a first open end 63 and a second open end 64. The second end is adapted to be sealed by a removable sealing member 5, and the needle shield 60 comprises an inner surface 61 and an outer surface 62. Furthermore, the needle shield comprises a tubular portion (68) defining a central cylindrical axis of the needle shield, and on FIG. 1(a) the cylindrical axis of the needle shield is collinear with the cylindrical axis (z) of the needle assembly.

The needle hub 10 comprises a shield connecting portion 51 adapted for forming a first connection portion for a frangible connection 53 to the needle shield 60, and likewise the needle shield 60 comprises a corresponding hub connecting portion 52 adapted for forming a second connection portion for the frangible connection 53 to the needle hub 10.

The needle assembly, shown in FIG. 1(a), is shown in an integral configuration, wherein the central cylindrical axes of the needle hub 10 and the needle shield 60 are collinearly aligned, and the first end 63 of the needle shield 60 is covered or partly covered by the needle hub 10. In the integral configuration, the frangible connection 53 forms an integral connection between the shield connecting portion 51 and the hub connecting portion 52.

By applying a torque or an axial force the needle assembly can be changed to a broken configuration, wherein the frangible connection 53 is broken. In the broken configuration, the needle hub 10 and needle shield 60 are two separate parts, and the needle shield 60 can be removed from the needle hub 10, by pulling the hub and the shield in opposite directions relative to each other. This situation is illustrated in FIG. 1(d).

The needle shield 60 is adapted to provide a clearance 95 between the inner surface 61 of the needle shield 60 and the cylindrical axis (z), and the clearance is adapted to minimize the risk for the needle shield 60 interfering with a received needle cannula 90. During use, there is a reduced risk of the needle shield interfering with a zone around the central axis, and as the needle to be received occupies a portion of the zone around the central axis, the described needle assembly enables a design reducing the risk of bending the needle.

Due to the second opening of the needle shield it is possible, in a one-part injection moulded needle assembly to provide a clearance between the needle and the inner surface of the needle shield. In other words, the second opening of the needle shield allows the forming of a clearance between the needle cannula and the inner surface of the needle shield. The second opening is formed by the mould insert, and can be visually observed after the mould insert has been withdrawn.

A needle shield moulded with an open end is also more flexible than a needle shield moulded with a closed end, this will, as explained later, allow a design where some surface structures may be provided, e.g., a snap locking feature.

The needle assembly is provided as a one-part injection moulded part, and in a sealed integral configuration the needle assembly provides the function of a sterile container, which can be used for storage, and the needle shield is further adapted to be used as a needle shield, for the needle assembly in the broken configuration, which will be explained in further details later.

The shield connecting portion 51 is here illustrated as a surface portion occupying a portion of the outer surface of the needle hub, and the hub connecting portion 52 is illustrated as a surface portion occupying a portion of the surface of the needle shield. The connecting portions 51, 52 are defined by the mould used to form the needle assembly, and the connecting portions will be formed on each side of the small channel forming the frangible zone.

### Release of Mould Insert

The needle assembly is adapted to be manufactured by injection moulding, and in order to make a sufficient clearance 95 between the needle cannula and the needle shield, the needle shield is formed with an open end, which allows the release and ejection of a mould, wherein the mould forms the inner surface of the needle shield.

In a similar way, a mould forms the inner surface of the needle hub, and this mould is released and ejected through an open end of the hub. In order to be able to release a mould, the inner surfaces of the shield and the hub may be adapted accordingly to provide this function. This means that the surface may not comprise any shoulders or protrusions entrapping an internal volume, and thereby preventing the surface forming mould to be ejected. However, small shoulders, protrusions or other surface structures may be possible if the injection moulded structure is sufficiently flexible. Furthermore, the formation of a thread is also feasible, as the mould can be released by rotation.

FIG. 1(a) shows a further aspect of an embodiment of the needle assembly 1, wherein the needle shield 60 is adapted to release a mould insert through the opening at the second end 64, and wherein the inner surface 61 of the shield 60 has been formed by the mould insert. The inner surface 61 of the needle shield 60 defines a draft or tapered angle (Y) to ease the release of the mould insert. The draft angle is defined as an angle between the cylindrical axis and an axis along the inner surface of the injection moulded surface. FIG. 1(a) illustrates an exaggerated draft angel (Y). In preferred embodiments the draft angle (Y) is between 0.2 and 5 degrees.

If the mould, forming the inner surface of the needle shield was to be ejected through the open end of the hub, it would not be possible to provide a needle assembly with an inner diameter larger than an inner diameter of the through hole in the hub. This is possible for the embodiments described in this application, as the mould forming the inner surface of the needle shield is released or ejected through an open end of the needle shield instead of the end covered or partly covered by the needle hub.

FIG. 1(a) shows a further aspect of an embodiment of the needle assembly 1, wherein the needle shield 60 comprises a first inner diameter at the opening of the first end 63, wherein the first inner diameter of the needle shield 60 is larger than an inner diameter of the through hole 48 of the needle hub 10, and thereby providing a needle assembly 1, which reduces the risk of bending a received needle 90. As explained, this design is enabled by the open end of the needle shield.

If the needle assembly is provided with a glue reservoir 49 colinearly aligned with the through hole 48. The inner surface of the glue reservoir defines a largest inner diameter at the distal end 14 of the needle hub 10. In the illustrated embodiments, the first inner diameter of the needle shield 60 is larger than the largest inner diameter defined by the inner surface of the glue reservoir 49.

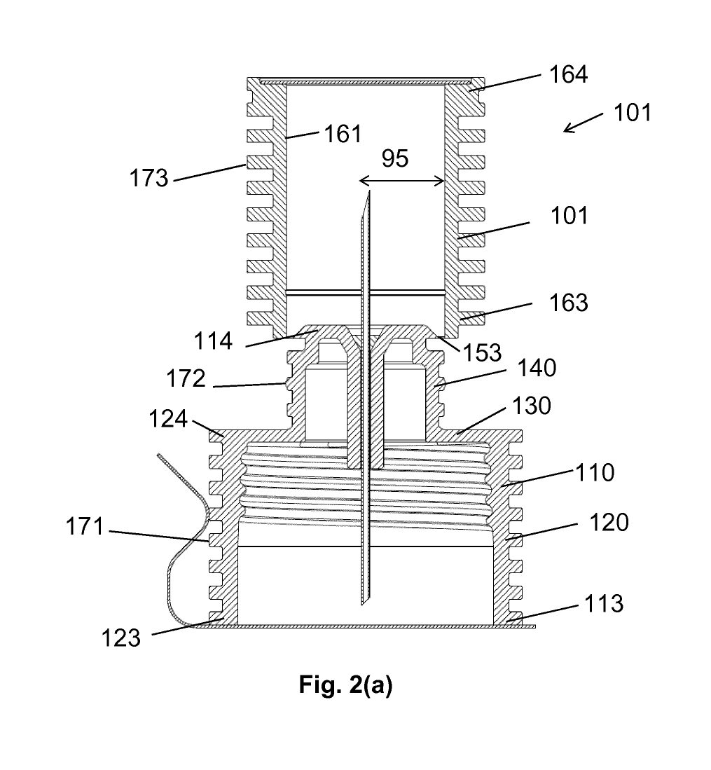

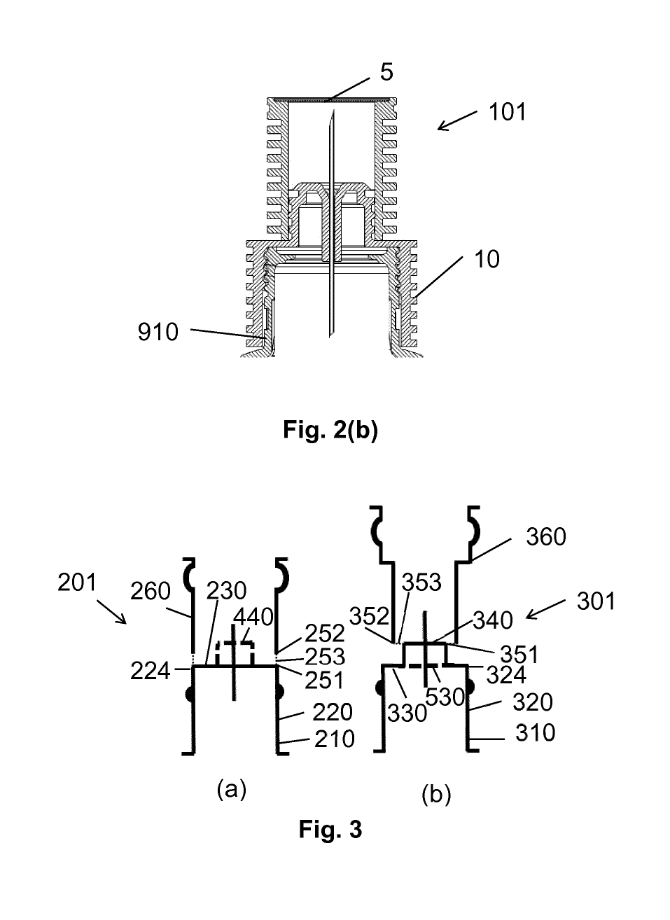

FIGS. 2(a), 3(a) and 3(b) shows alternative embodiments of the needle assembly 101, 201, 301 with features corresponding to the features and functions described above for needle assembly 1. Similar features are provided with similar numbers, but a higher number of details are indicated with reference numbers for the needle assemblies 1 and 101 compared to 201 and 301.

### Slim Shield Design

In another aspect it may be desirable also to provide a needle shield being as slim as possible, and thereby allowing the shield to fit under a cap for a medical device, in a situation where it is desired to leave the needle assembly mounted on a needle mount and with the cap mounted on the medical device. Thereby, the needle is both protected by the cap and the needle shield. In some embodiments of the needle assembly 1, 101, 301 this feature is enabled, by allowing the needle shield to be designed with an outer diameter less than the outer diameter of the needle hub.

FIGS. 1, 2 and 3(b) shows an aspect of the needle shield 1, 101, 301, wherein the tubular portion 20, 120, 320 of the needle hub 10, 110, 310 defines a distal outer diameter at the distal end 24, 124, wherein the shield connecting portion 51, 351 is a portion of the central hub portion 30, 130, 330 and defines a shield connecting diameter. The shield connecting diameter is smaller than the distal outer diameter of the tubular portion 20, 120, 320. In the illustrated embodiments the shield connecting portion is shown to be positioned on a projecting portion 40, 140, 340, as a preferred embodiment, but it could also be positioned on the skirt connecting portion 31, and obtain a similar effect, i.e., to allow a more slim needle shield.

FIGS. 1, 2 and 3(b) shows an aspect of the needle assembly 1, 101, 301, wherein the needle shield 60, 160, 360 defines a first outer diameter at the first end 63, 163. The embodiments provides a needle assembly, wherein the first outer diameter of the needle shield 60, 160, 360 is smaller than the distal outer diameter of the tubular portion 20, 120, 320 of the needle hub 10, 110, 310.

FIG. 3(a) shows an aspect of the embodiment 201, wherein the tubular portion 220 of the needle hub 210 defines a distal outer diameter at the distal end 224, wherein the shield connecting portion 251 is a portion of the tubular hub portion 220 and defines a shield connecting diameter, and wherein the shield connecting diameter is equal to or smaller than the distal outer diameter of the tubular portion 220. By nature, the shield connecting diameter cannot be larger than the distal outer diameter of the tubular portion, as it then would be disconnected from the tubular portion. Similarly to the way the shield connecting portion defined a shield connecting diameter, the hub connecting portion 252 could define a hub connecting diameter, with a diameter equal to the shield connecting diameter. Consequently, the hub connecting diameter would be equal to or smaller than the outer diameter of the tubular portion, and an opening defined by the hub connecting diameter would not be able to receive the distal end of the tubular portion 220.

### Projecting Portion, Actuation Design

FIGS. 1, 2 and 3(b) shows another aspect of the needle assembly 1, 101, 301, wherein the central hub portion 30, 130, 330 comprises a projecting hub portion 40, 140, 340 having a tubular portion 41 and a central portion 45 covering a distal end of the tubular portion 41. The through hole 48 and the shield connecting portion 51 is positioned on a distal portion of the projecting hub portion 40, 140, 340. The projecting portion provides an additional longitudinal surface on the tubular portion of the projecting portion, and this surface allows the provision of different functionalities relating to attachment between hub and shield.

In a further aspect, as shown for the needle assembly 1, 101, 301, the shield connecting portion 51, 351 is positioned on an outer surface of the tubular portion 41 of the projecting hub portion 40, 140, 340, wherein the opening at the first end 63, 163 of the needle shield is adapted to receive a portion of the projecting hub portion 40, 140, 340. The needle assembly 1, 101, 301 is adapted to be changed from the integral configuration to the broken configuration by pushing the needle shield 60, 160, 360 in the direction of the needle hub 10, 110, 310. In this way the projecting portion provides an attachment surface for the first end of the needle shield, as well as a structure that enables an easy change of the needle assembly from the integral configuration to the broken configuration.

In a further aspect of the embodiment 101 the opening at the first end 163 of the needle shield 160 is adapted to be pressure fitted onto the projecting hub portion 140, for the needle assembly being in the broken configuration. This is shown in FIG. 2(a).

### Second End of Needle Shield

FIGS. 1, 3(a) and 3(b) shows another aspect of the embodiments 1, 201, 301, wherein the opening at the second end 64 of the needle shield 60, 260, 360 is adapted to receive a portion of the needle hub 10, 210, 310. The inner surface 61 at the second end 64 of the needle shield 60 is adapted to match an outer surface of the needle hub 10, 210, 310, and the needle shield 60, 260, 360 is adapted to be fitted onto the needle hub 10, 210, 310, by receiving the portion of the needle hub 10, 210, 310 through the opening at the second end 64 of the needle shield 60, 260, 360.

FIGS. 1, 3(a) and 3(b) shows a further aspect of the embodiments 1, 201, 301, wherein the inner surface 61 of the needle shield 60, 260, 360 forms a snap connecting member 65, and the outer surface of the needle hub 10, 210, 310 comprises a corresponding snap connecting member 41. The needle shield 60, 260, 360 is thereby adapted to be snapped onto the needle hub 10, 210, 310, for the needle assembly in the broken configuration.

In a further aspect as shown for the embodiment 1, the snap connecting member 42 can be positioned on an outer surface of the tubular portion 41 of the projecting hub portion 40.

In an alternative embodiment 201, 301 the snap connecting member is positioned on an outer surface of the tubular portion 220, 320.

In another embodiment as illustrated for the needle assembly 1, see FIG. 1(a), the cylindrical axis (z) and the inner surface 61 of the needle shield 60 defines a draft angle Y. The outer surface of the tubular portion 41 of the projecting hub portion 40 is adapted to match the draft angle (Y). In this way, the connection between the second end of the needle shield portion and the projecting hub portion is optimized.

### Increased Welding Area

In another embodiment as illustrated for the needle assembly, 1, 101, 201, 301, the needle hub 10, 110, 210, 310 comprises a sealing flange 15 positioned at the proximal end 23, which sealing flange is adapted to receive a sealing member 2, which is to be welded onto a surface. Due to the slim design of the needle hub, having the function of sterile container, the flange can have a larger sealing area compared to traditional needle container, and still be accommodated under a cap for a medical device, e.g., a pen cap.

In another embodiment as illustrated for the needle assembly 1, 101, 201, 301 the needle shield 60, 160, 260, 360 comprises a sealing flange 66 positioned at the second end 64, 164 of needle shield 60, 160, 260, 360, wherein the sealing flange is adapted to receive a sealing member 5, which is to be welded onto a surface. Due to the slim design of the needle shield, the flange can have a larger sealing area compared to a traditional needle container, and still be accommodated under a cap for a medical device, e.g., a pen cap. When the second end of the needle cap is used to shield the needle, for the needle assembly in the broken configuration, the second end will be upside-down compared to a situation where the needle assembly is in the integral configuration. Caps for medical devices having a closed and an open end are typically tapering towards the closed end, and an inner diameter of the cap is therefore increasing towards the open end. Therefore, the second end of the needle shield can be larger than the first end of the needle shield, if the needle shield is to be accommodated under a cap for a medical device, in an upside-down orientation, i.e., when the second end of the shield is oriented towards the open end of the cap.

Alternatively, the sealing member is applied by heat sealing, and the required area for sealing can be even smaller. In this case the sealing member could be made of Tyvec®.

### Side Actions

In another embodiment, as illustrated for the needle assembly 1, 101, 301, the outer surface of the needle assembly comprises two or more radially extending structures 67, 31, 171, 172, 173. The at least two radially extending structures confine a volume, which is closed in the cylindrical direction. The volume can have an opening in the radial direction, and thereby allow a mould to be pulled out in this direction. The radially extending structures can be structures 67, 31 of the shield 60 or the hub 10, or it can be additional gripping structures 171, 173, providing a better grip during handling.

Therefore, the described embodiments may be produced by injection moulded where the mould is pulled out in a cylindrical and in a radial direction.

### Handling of the Needle Assembly

FIGS. 1(b) to 1(d) illustrates how the needle assembly may be handled. In FIG. 1(b) the needle assembly 1 is shown in an integral configuration. In the shown embodiment the first sealing member 2 and the second sealing member 5 are connected, and may be separated in the separation zone 4.

In FIG. 1(c) the sealing members are separated in the separation zone 4, and the first sealing member 2 is removed from the proximal opening of the hub 10. Hereafter, the needle assembly is mounted on the needle mount of the injection device 900.

To arrive with the assembly 1 shown in FIG. 1(d) the needle shield 60 is turned relative to the needle hub 10, whereby the frangible zone is broken, and the shield is removed. Alternatively, the needle shield is pushed towards the needle hub until a surface of the needle shield abuts a surface of the needle hub. When the latter method is used for breaking the frangible zone, the longitudinal movement should not exceed the distance defined between the second sealing member and the needle tip.

In FIG. 1(e) the needle shield is turned upside-down, and the second end is fitted onto the projecting portion 40. Hereafter, a pen cap may be mounted if the medical device is an injection device of the pen type. The pen cap can accommodate the needle assembly, when the shield is mounted through its second end, and if the first end is designed sufficiently slim or narrow.

FIG. 2(b) shows the needle assembly 101 in the broken configuration, and wherein the first end of the needle shield has been fitted onto the projecting portion 140. In this configuration the needle shield may be designed to rotationally lock to the needle hub, whereby the shield can be used to mount and demount the needle hub.

Although FIG. 3(a) illustrates an embodiment of a needle assembly without a projecting hub portion, an alternative embodiment could be realized with a hub projecting portion. This is indicated with dashed line 440. Similarly, although the embodiment illustrated in FIG. 3(b) provides a projecting hub portion, an embodiment with a more plane central portion is illustrated with a dashed line (530). In such an embodiment a shield connecting portion would be positioned on the outer surface of the plane central portion.

### List of Embodiments

1. A needle assembly (1, 101, 201, 301) for a medical device (900), wherein the needle assembly (1,101, 201, 301) is having a central cylindrical axis (z) and a radial axis (r) defining a cylindrical and a radial direction, and wherein the needle assembly (1, 101, 201, 301) comprises:

a needle hub (10, 110, 210, 310) adapted for receiving a needle cannula (90), wherein the needle hub (10, 110, 210, 310) is having an inner (11) and an outer surface (12), a proximal (13, 113) and a distal end (14, 114), and wherein the needle hub (10, 110, 210, 310) comprises:

a skirt portion (20, 120, 220, 320) comprising:
a connecting member (29) for removably connecting the needle hub (10, 110, 210, 310) to a needle mount (910) of the medical device (900),
a proximal end (23, 123) defining an opening adapted to be sealed by a removable sealing member (2), and a distal end (24, 124, 224, 324) defining an opening adapted to be covered by a central hub portion (30, 130, 230, 330),

the central hub portion (30, 130, 230, 330) comprising:
a through hole (48) colinearly aligned with the cylindrical axis (z), and adapted to receive the needle cannula (90),
a skirt connecting portion (31) connected to a distal portion of the skirt portion (20, 120, 220, 320), and

a needle shield (60, 160, 260, 360) having a first open end (63, 163) and a second open end (64, 164), and wherein the second end is adapted to be sealed by a removable sealing member (5), and wherein the needle shield (60, 160) comprises an inner (61) and an outer surface (62),

wherein the needle hub (10, 110, 210, 310) comprises a shield connecting portion (51, 251, 351) adapted for forming a first connection portion for a frangible connection (53, 153, 253, 353) to the needle shield (60, 160, 260, 360), and wherein the needle shield (60, 160, 260, 360) comprises a corresponding hub connecting portion (52, 252, 352) adapted for forming a second connection portion for the frangible connection (53, 253, 353) to the needle hub (10, 210, 310),

wherein the needle assembly is adapted to be changed from:
an integral configuration, wherein the needle hub (10, 110, 210, 310) and the needle shield (60, 160, 260, 360) are collinearly aligned, wherein the first end (63, 163) of the needle shield (60, 160, 260, 360) is covered by the needle hub (10, 110, 210, 310), and wherein the frangible connection (53, 153, 253, 353) forms an integral connection between the shield connecting portion (51, 251, 351) and the hub connecting portion (52, 252, 352), to
a broken configuration, wherein the frangible connection (53, 153, 253, 353) is broken, and whereby the needle hub (10, 110, 210, 310) and needle shield (60, 160, 260, 360) are two separate parts, and whereby the needle shield (60, 160, 260, 360) is removable from the needle hub (10, 110, 210, 310),

whereby the needle shield (60, 160, 260, 360) is adapted to provide a clearance (95) between the inner surface (61) of the needle shield (60, 160, 260, 360) and the cylindrical axis (z), and whereby the clearance (95) is adapted to minimize the risk for the needle shield (60, 160,260, 360) interfering with a received needle cannula (90).

### Release of Mould Insert

2. A needle assembly (1, 101, 201, 301) according to any of the previous embodiments, wherein

the needle shield (60, 160, 260, 360) is adapted to release a mould insert through the opening at the second end (64, 164), and wherein the inner surface (61, 161) of the shield (60, 161) has been formed by the mould insert.

3. A needle assembly (1, 101, 201, 301) according to any of the previous embodiments, wherein

the cylindrical axis (z) and the inner surface (61, 161) of the needle shield (60, 160, 260, 360) defines a draft angle (Y) to ease the release of the mould insert.

4. A needle assembly (1, 101, 201, 301) according to embodiment 3, wherein the draft angle (Y) is

between 0.2 and 5 degrees.

5. A needle assembly (1, 101, 201, 301) according to any of the previous embodiments, wherein

the inner surface (61, 161) of the needle shield (60, 160, 260, 360) defines a closed curve on a plane defined by a cylindrical coordinate (zp) along the cylindrical axis, wherein the cylindrical coordinate is defined between the first and the second end of the needle shield (60, 160), wherein the closed curve defines a diameter (Dzp) dependent on the cylindrical coordinate (zp), and wherein the diameter (Dzp) of the closed curve substantially increases in a direction from the first to the second end, whereby no volume is entrapped in the needle shield (no under-cut).

6. A needle assembly (1, 101, 201, 301) according to any of the previous embodiments, wherein

the needle shield (60, 160, 260, 360) comprises a first inner diameter at the opening of the first end (63, 163), wherein the first inner diameter of the needle shield (60, 160) is larger than an inner diameter of the through hole (48) of the needle hub (10), and thereby providing a needle assembly (1, 101, 201, 301) reducing the risk of bending a received needle (90).

7. A needle assembly (1, 101, 201, 301) according to embodiment 6, wherein the needle hub (10,

110, 210, 310) comprises a glue reservoir (49) aligned with the through hole (48), wherein an inner surface of the glue reservoir defines a largest inner diameter at the distal end (14, 114) of the needle hub (10, 110), and wherein the first inner diameter of the needle shield (60, 160, 260, 360) is larger than the largest inner diameter defined by the inner surface of the glue reservoir (49).

### Slim Shield Design

8. A needle assembly (1, 101, 301) according to any of the previous embodiments, wherein the

tubular portion (20, 120, 320) of the needle hub (10, 110, 310) defines a distal outer diameter at the distal end (24, 124), wherein the shield connecting portion (51, 351) is a portion of the central hub portion (30, 130, 330) and defines a shield connecting diameter, and wherein the shield connecting diameter is smaller than the distal outer diameter of the tubular portion (20, 120, 320).

9. A needle assembly (1, 101, 301) according to embodiment 8, wherein the needle shield (60,

160) defines a first outer diameter at the first end (63, 163), and wherein the first outer diameter of the needle shield (60, 160, 360) is smaller than the distal outer diameter of the tubular portion (20, 120, 320) of the needle hub (10, 110, 310).

10. A needle assembly (201) according to any of the embodiments 1-7, wherein the tubular portion

(201) of the needle hub (210) defines a distal outer diameter at the distal end (224), wherein the shield connecting portion (251) is a portion of the tubular hub portion (220) and defines a shield connecting diameter, and wherein the shield connecting diameter is equal to or larger than the distal outer diameter of the tubular portion (220).

### Projecting Portion, Actuation Design

11. A needle assembly according to embodiment 10, wherein the central hub portion comprises a

projecting hub portion having a tubular portion and a central portion covering a distal end of the tubular portion, wherein the through hole and the shield connecting portion is positioned on a distal portion of the projecting hub portion.

12. A needle assembly (1, 101, 301) according to any of the embodiments 1-9, wherein the central hub portion (30, 130, 330) comprises a projecting hub portion (40, 140, 340) having a tubular portion (41) and a central portion (45) covering a distal end of the tubular portion (41), wherein the through hole (48) and the shield connecting portion (51) is positioned on a distal portion of the projecting hub portion (40, 140, 340).

13. A needle assembly (1, 101, 301) according to any of embodiments 1-9, wherein the shield

connecting portion (51, 351) is positioned on an outer surface of the tubular portion (41) of the projecting hub portion (40, 140, 340), wherein the opening at the first end (63, 163) of the needle shield is adapted to receive a portion of the projecting hub portion (40, 140, 340), and wherein the needle assembly (1, 101, 301) is adapted to be changed from the integral configuration to the broken configuration by pushing the needle shield (60, 160, 360) in the direction of the needle hub (10, 110, 310).

14. A needle assembly (101) according to embodiment 13, wherein the opening at the first end (163)

of the needle shield (160) is adapted to be pressure fitted onto the projecting hub portion (140), for the needle assembly being in the broken configuration.

### Second End of Needle Shield Portion

15. A needle assembly (1, 201, 301) according to any of embodiments 1-13, wherein the opening at

the second end (64) of the needle shield (60, 260, 360) is adapted to receive a portion of the needle hub (10, 210, 310), wherein the inner surface (61) at the second end (64) of the needle shield (60) is adapted to match an outer surface of the needle hub (10, 210, 310), and wherein the needle shield (60, 260, 360) is adapted to be fitted onto the needle hub (10, 210, 310), by receiving the portion of the needle hub (10, 210, 310) through the opening at the second end (64) of the needle shield (60, 260, 360).

16. A needle assembly (1, 201, 301) according to any of the previous embodiments, wherein the inner surface (61) of the needle shield (60, 260, 360) forms a snap connecting member (65), and wherein an outer surface of the needle hub (10, 210, 310) comprises a corresponding snap connecting member (41), and wherein the needle shield (60, 260, 360) is adapted to be snapped onto the needle hub (10, 210, 310), for the needle assembly in the broken configuration.

17. A needle assembly (1) according to embodiment 16, wherein the snap connecting member (42) is positioned on an outer surface of the tubular portion (41) of the projecting hub portion (40).

18. A needle assembly (201, 301) according to embodiment 16, wherein the snap connecting member is positioned on an outer surface of the tubular portion (220, 320).

19. A needle assembly (1) according to any of the embodiments 1-17, wherein the cylindrical axis (z) and the inner surface (61) of the needle shield (60) defines a draft angle (Y), and wherein the outer surface of the tubular portion (41) of the projecting hub portion (40) is adapted to match the draft angle (Y), whereby the connection between the second end of the needle shield portion and the projecting hub portion is optimized.

### Increased Welding Area

20. A needle assembly (1, 101, 201, 301) according to any of the previous embodiments, wherein

the needle hub (10, 110, 210, 310) comprises a sealing flange (15) positioned at the proximal end (23), which sealing flange is adapted to receive a sealing member (2), which is to be welded onto a surface.

21. A needle assembly (1, 101, 201, 301) according to any of the previous embodiments, wherein

the needle shield (60, 160, 260, 360) comprises a sealing flange (66) positioned at the second end (64, 164) of needle shield (60, 160, 260, 360), wherein the sealing flange is adapted to receive a sealing member (5), which is to be welded onto a surface.

### Side Actions

22. A needle assembly (1, 101, 301) according to any of the previous embodiments, wherein the outer surface of the needle assembly comprises 2 or more radially extending structures (67, 31, 171, 172, 173), wherein the at least two radially extending structures confines a volume which is closed in the cylindrical direction.

23. A needle assembly according to embodiment 22, wherein the radially extending structures comprises gripping structures (171, 173).

24. A needle assembly (1, 201, 301) for a medical device (900) comprising:

a needle cannula (90) and a needle hub (10, 210, 310), wherein the needle hub (10, 210, 310) is having a skirt portion (20, 220, 320) and a central portion (30, 230, 330), wherein the needle cannula (90) is received in the central portion (30, 230, 330),

a tubular shield (60, 260, 360) adapted to shield a distal end of the needle cannula (90), and to cover a portion of the needle hub (10, 210, 310),

a frangible zone (53, 253, 353),

the needle hub (10, 210, 310) comprises a distal portion with a distal end (14), and a proximal portion with a proximal end (13) having a first opening surrounded by a first sealing surface adapted to attach to a first sealing member (2),

wherein the distal portion is positioned on the central portion (30, 230, 330), and wherein the proximal portion of the needle hub (10, 210, 310) is a portion of the skirt portion (20, 220, 320),

wherein the needle shield (60, 260, 360) comprises a first portion having a first end (63) and a second portion having a second end (64),

wherein the second end (64) is having a second opening surrounded by a second sealing surface adapted to attach to a second sealing member (5), and wherein the second opening is adapted to be fitted onto a distal portion of hub (10, 210, 310), and thereby being adapted to shield the needle cannula (90),

wherein the needle assembly (1, 201, 301) is adapted to be changed from:

an initial shielded configuration, wherein the frangible zone (53, 253, 353) directly connects the distal portion of the hub and the first portion of the needle shield (60, 260, 360), and a broken configuration, wherein the frangible zone (53, 252, 353) is broken and the needle shield (60, 260, 360) is removable from the needle hub (10, 210, 310).

25. A medical system comprising:

a needle assembly (1, 101, 201, 301) according to any of the previous embodiments, and

a medical device (900) comprising:
a mounting portion,
a device cap adapted to cover the mounting portion (910), and
wherein the mounting portion comprises a needle mount (910) and a device cap mount,

wherein the medical system is configured to be in a state:
wherein the needle assembly is mounted on the needle mount (910),
wherein the needle assembly is in a broken configuration,
wherein the needle shield (60, 160, 260, 360) is fitted onto a distal portion of the needle hub (10, 110, 210, 310),
wherein the device cap is mounted on the cap mount, and
wherein the device cap accommodates the needle assembly.

### Method of Handling

26. A method of handling a needle assembly according to any of the embodiments 1-24, wherein the method comprises:
providing a needle assembly (1, 101, 201, 301) according to any of the embodiments 1-24,
changing the needle assembly from an integral configuration to a broken configuration.

27. A method according to embodiment 26 further comprising the step of fitting the needle shield

(60, 160, 260, 360) onto the needle hub (10, 110, 210, 310), when the needle assembly (1, 101, 201, 301) is in the broken configuration.

28. A method according to embodiment 27, wherein a portion of the needle hub (10, 210, 310) is

received through the second end (64) of the needle shield (60, 260, 360), and the needle shield is fitted onto the needle hub (10, 210, 310).

### Method of Producing a Needle Assembly

29. A method of producing a needle assembly (1, 101, 201, 301) according to any of the embodiments 1-24, wherein the method comprises:
providing an injection mould for forming the needle assembly
filling the injection mould and thereby forming the needle assembly
pulling the injection mould in a first direction, and thereby separating the mould from a first portion of the needle assembly
separating the injection mould along a second direction, and thereby separating the mould from a second portion of the needle assembly.

In the above description of the preferred embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

While certain features of the invention have been illustrated and described herein, many modifications, substitutions, changes, and equivalents will now occur to those of ordinary skill in the art. It is, therefore, to be understood that the appended embodiments are intended to cover all such modifications and changes as fall within the true spirit of the invention.

1. A needle assembly for a medical device, wherein the needle
assembly is having a central cylindrical axis (z) and a radial axis (r) defining a cylindrical and a radial direction, and wherein the needle assembly comprises:
a needle hub adapted for receiving a needle cannula, wherein the needle hub is having an inner and an outer surface, a proximal and a distal end, and wherein the needle hub comprises:
a skirt portion comprising:
a connecting member for removably connecting the needle hub to a needle mount of the medical device,
a proximal end defining an opening adapted to be sealed by a removable sealing member, and a distal end defining an opening adapted to be covered by a central hub portion,
the central hub portion comprising:
a through hole defining a central cylindrical axis collinearly aligned with the cylindrical axis (z) of the needle assembly, and adapted to receive the needle cannula,
a skirt connecting portion connected to a distal portion of the skirt portion, and
a needle shield having a first end defining a first opening and a second end defining a second opening, and wherein the second opening is adapted to be sealed by a removable sealing member, wherein the needle shield comprises a tubular portion defining a central cylindrical axis of the needle shield, wherein the needle shield comprises an inner and an outer surface,
wherein the needle hub comprises a shield connecting portion adapted for forming a first connection portion for a frangible connection to the needle shield, and wherein the needle shield comprises a corresponding hub connecting portion adapted for forming a second connection portion for the frangible connection to the needle hub,
wherein the needle assembly is adapted to be changed from:
an integral configuration, wherein the central cylindrical axes of the needle hub and the needle shield are collinearly aligned with the cylindrical axis, wherein the first opening defined at the first end of the needle shield is covered by the needle hub, and wherein the frangible connection forms an integral connection between the shield connecting portion and the hub connecting portion, and wherein a clearance is provided between the needle shield to
a broken configuration, wherein the frangible connection is broken, and whereby the needle hub and needle shield are two separate parts, and whereby the needle shield is adapted to be removable from the needle hub by pulling the needle hub and needle shield in opposite directions,
whereby the needle shield is adapted to provide the clearance between the inner surface of the needle shield and the cylindrical axis (z), and whereby the clearance is adapted to minimize the risk for the needle shield interfering with a received needle cannula.

2. The needle assembly according to claim 1, wherein the needle shield is adapted to release a mould insert through the opening at the second end, and wherein the inner surface of the shield has been formed by the mould insert.

3. The needle assembly according to claim 1, wherein the inner surface of the needle shield defines a closed curve on a plane defined by a cylindrical coordinate (zp) along the cylindrical axis, wherein the cylindrical coordinate is defined between the first and the second end of the needle shield, wherein the closed curve defines a diameter (Dzp) dependent on the cylindrical coordinate (zp), and wherein the diameter (Dzp) of the closed curve substantially increases in a direction from the first to the second end, whereby no volume is entrapped in the needle shield.

4. The needle assembly according to claim 1, wherein the needle shield comprises a first inner diameter at the opening of the first end, wherein the first inner diameter of the needle shield is larger than an inner diameter of the through hole of the needle hub, and thereby providing a needle assembly reducing the risk of bending a received needle.

5. The needle assembly according to claim 1, wherein the tubular portion of the needle hub defines a distal outer diameter at the distal end, wherein the shield connecting portion is a portion of the central hub portion and defines a shield connecting diameter, and wherein the shield connecting diameter is smaller than the distal outer diameter of the tubular portion.

6. The needle assembly according to claim 5, wherein the needle shield defines a first outer diameter at the first end, and wherein the first outer diameter of the needle shield is smaller than the distal outer diameter of the tubular portion of the needle hub.

7. The needle assembly according to claim 1, wherein the central hub portion comprises a projecting hub portion having a tubular portion and a central portion covering a distal end of the tubular portion, wherein the through hole and the shield connecting portion is positioned on a distal portion of the projecting hub portion.

8. The needle assembly according to claim 1, wherein the shield connecting portion is positioned on an outer surface of the tubular portion of the projecting hub portion, wherein the opening at the first end of the needle shield is adapted to receive a portion of the projecting hub portion, and wherein the needle assembly is adapted to be changed from the integral configuration to the broken configuration by pushing the needle shield in the direction of the needle hub.

9. The needle assembly according to claim 8, wherein the opening at the first end of the needle shield is adapted to be pressure fitted onto the projecting hub portion, for the needle assembly being in the broken configuration.

10. The needle assembly according to claim 1, wherein the opening at the second end of the needle shield is adapted to receive a portion of the needle hub, wherein the inner surface at the second end of the needle shield is adapted to match an outer surface of the needle hub, and wherein the needle shield is adapted to be fitted onto the needle hub, by receiving the portion of the needle hub through the opening at the second end of the needle shield.

11. The needle assembly according to claim 1, wherein the inner surface of the needle shield forms a snap connecting member, and wherein an outer surface of the needle hub comprises a corresponding snap connecting member, and wherein the needle shield is adapted to be snapped onto the needle hub, for the needle assembly in the broken configuration.

12. The needle assembly according to claim 11, wherein the snap connecting member is positioned on an outer surface of the tubular portion of the projecting hub portion.

13. The needle assembly according to claim 1, wherein the cylindrical axis (z) and the inner surface of the needle shield defines a draft angle (Y) enabling removal of a mold insert through the second end of the shield, and wherein the outer surface of the tubular portion of the projecting hub portion is adapted to match the draft angle (Y) and for forming a pressure fitted connection, whereby the connection between the second end of the needle shield portion and the projecting hub portion is optimized for forming a pressure fitted connection.

14. The needle assembly according to claim 1, wherein the needle hub comprises a sealing flange positioned at the proximal end, which sealing flange is adapted to receive a sealing member, which is to be welded onto a surface.

15. The needle assembly according to claim 1, wherein the needle shield comprises a sealing flange positioned at the second end of needle shield, wherein the sealing flange is adapted to receive a sealing member, which is to be welded onto a surface.
